(19) United States
(12) Patent Application Publication
(10) Pub. No.: US 2026/0284087 A1
(43) Pub. Date: Sep. 24, 2026

(54) USES OF CANNABIDIOL (CBD) AND ADVANCED METABOLIC SYNDROME

(71) Applicant: THE REGENTS OF THE UNIVERSITY OF CALIFORNIA, Oakland, CA (US)

(72) Inventors: Rudy M. ORTIZ, Merced, CA (US); Jessica WILSON, Merced, CA (US)

(73) Assignee: THE REGENTS OF THE UNIVERSITY OF CALIFORNIA, Oakland, CA (US)

(21) Appl. No.: 19/126,122

(22) PCT Filed: Nov. 2, 2023
(86) PCT No.: PCT/US2023/078564

§ 371 Date: Apr. 30, 2025

Related U.S. Application Data
(60) Provisional application No. 63/421,858, filed on Nov. 2, 2022.

(51) Int. Cl. A61K 31/00; A61P 3/00

(52) U.S. Cl. CPC A61K 31/658; A61P 3/00

(57) ABSTRACT

Aspects of the present disclosure relate to the use of CBD for treating metabolic dysfunction and associated conditions.

### BACKGROUND

This application claims priority of U.S. Provisional Patent Application No. 63/421,858 filed Nov. 2, 2022, which is hereby incorporated by reference in its entirety.

### I. FIELD OF THE INVENTION

This invention relates generally to the fields of pharmacology and medicine.

### II. Background

Metabolic syndrome (MetS) is estimated to affect over 1 billion people globally and is a precursory condition of lethal conditions, including cardiovascular disease. Cannabis has been implicated to improve risk factors of MetS, including obesity and dyslipidemia, but the intoxicating properties of whole plant consumption limit such application. Cannabidiol (CBD) is an abundant, non-intoxicating constituent of Cannabis sativa, which ameliorates the same risk factors when administered alone across various models of isolated risk factors, such as elevated arterial pressure, adiposity, glucose intolerance, and dyslipidemia. Of those risk factors, the loss of brown adipose tissue (BAT), which is an indicator for impaired overall metabolism, has not been investigated. Therefore, there exists a need for therapeutics for treating MetS and associated conditions.

### SUMMARY

Disclosed herein are methods and compositions for treating metabolic dysfunction and conditions associated with metabolic dysfunction. In some aspects, a method of treating metabolic dysfunction or a condition associated with metabolic dysfunction comprises administering a therapeutically effective amount of a composition comprising CBD to an individual in need thereof. In some aspects, the condition associated with metabolic dysfunction is selected from obesity, insulin resistance, reduced glucose tolerance, and metabolic syndrome. In some aspects, the individual has been diagnosed with metabolic dysfunction or a condition associated with metabolic dysfunction. In some aspects, the composition decreases body mass.

Some aspects of the disclosure are directed to a method of reducing body mass in an individual comprising administering to the individual a therapeutically effective amount of a composition comprising CBD. In some aspects, the reduced body mass comprises a reduction in abdominal fat. Some aspects of the disclosure are directed to a method of preserving brown adipose tissue in an individual comprising administering to the individual a therapeutically effective amount of a composition comprising CBD. In some aspects, the method increases ß-aminoisobutyric acid (BAIBA). In certain aspects, the individual has, or is at risk of having, metabolic syndrome.

In some aspects, the decrease in body mass is a reduction in abdominal fat. In some aspects, the composition increases insulin receptor expression. In some aspects, the composition reduces circulating adiponectin. In some aspects, the composition increases circulating ghrelin. In some aspects, the composition reduces plasma triglycerides. In some aspects, the composition increases lipid metabolism.

In some aspects, the CBD is a pharmaceutically acceptable salt, enantiomer, diastereomer, or prodrug thereof. In some aspects, the composition is administered orally, intraadiposally, intraarterially, intraarticularly, intracranially, intradermally, intralesionally, intramuscularly, intraperitoneally, intrapleurally, intranasally, intraocularally, intrapericardially, intraprostaticaly, intrarectally, intrathecally, intratumorally, intraumbilically, intravaginally, intravenously, intravesicularlly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof. In some aspects, the composition is administered to the subject at least two, three, four, five, six, seven, eight, nine or ten times. In some aspects, the CBD is administered at a dosage of at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, or 20 mg/kg. Furthermore, such doses can be administered at multiple times during a day, including 1, 2, 3, 4, 5, 6, 7, 8, 9, 10 or more doses per day, and/or on multiple days, weeks, or months. In certain instances, it will be desirable to have multiple administrations of the composition, e.g., 2, 3, 4, 5, 6 or more administrations. The administrations can be given every 1, 2, 3, 4, 5, 6, 7, 8, 9, 10, 11, 12, 13, 14 days, weeks, or months, including all ranges there between.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1E CBD preserves brown adipose tissue. (A-C) Representative images of peri-renal brown adipose tissue in LETO, OLETF, and OLETF treated with CBD, D) schematic of image field and E) mean pixel area normalized to LETO. *p<0.05 compared to LETO and †p<0.05 compared to OLETF by ANOVA with Tukey's HSD or student's t-test.

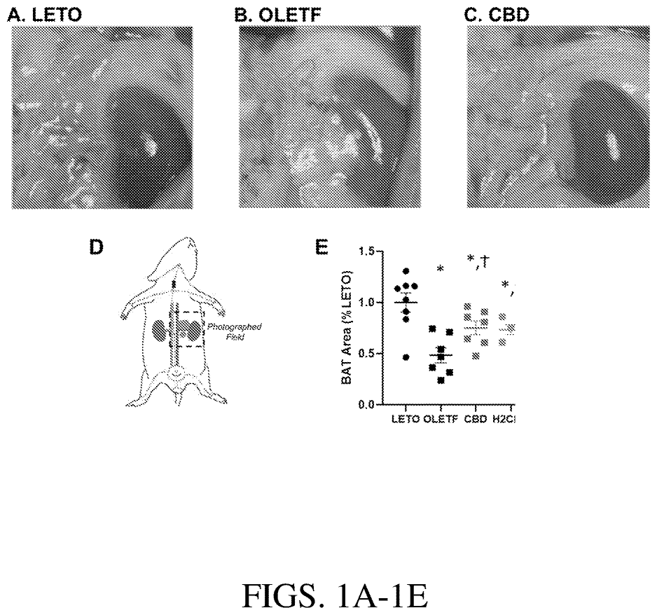

FIGS. 2A-2C CBD increases BAIBA. A) Metabolomic map showing metabolites with p<0.05 after Student's t-test, representing mean plasma peak intensity fold-changes between groups in metabolites with q<0.2, for OLETF vs. CBD, B) top 5 metabolites, associated pathway and change directionality listed by lowest p-value for OLETF vs. CBD and C) raw mean peak area of BAIBA. *p<0.05 compared to LETO and †p<0.05 compared to OLETF by ANOVA with Tukey's HSD or student's t-test.

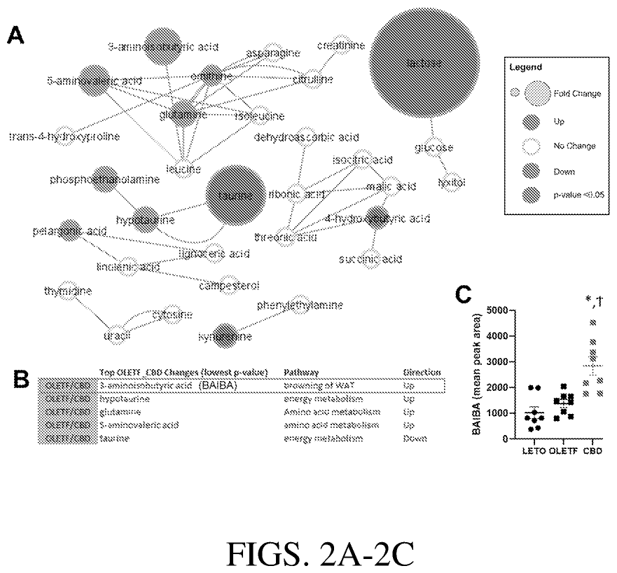

### DETAILED DESCRIPTION

The effects of cannabidiol (CBD) on MetS and adipose tissue were examined. Because of the explosion of interest in cannabis and cannabis constituents in older adults and the high incidence of MetS and cardiovascular disease of this population, a critical intersection of cannabis constituent use and advanced metabolic dysfunction exists.

I. Definitions The meaning of certain terms as intended is defined herein below.

“Metabolic syndrome” is a cluster of a cluster of biochemical and physiological abnormalities associated with the development of cardiovascular disease and type 2 diabetes.

“Individual, “subject,” and “patient” are used interchangeably and can refer to a human or non-human.

The terms “lower,” “reduced,” “reduction,” “decrease,” or “inhibit” are all used herein generally to mean a decrease by a statistically significant amount. However, for avoidance of doubt, “lower,” “reduced,” “reduction, “decrease,” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

Suitable pharmaceutically acceptable salts may also be formed by reacting the agents of the invention with an organic base such as methylamine, ethylamine, ethanolamine, lysine, ornithine and the like. Pharmaceutically acceptable salts include the salts formed between carboxylate or sulfonate groups found on some of the compounds of this invention and inorganic cations, such as sodium, potassium, ammonium, or calcium, or such organic cations a isopropylammonium, trimethylammonium, tetramethylammonium, and imidazolium.

It should be recognized that the particular anion or cation forming a part of any salt of this invention is not critical, so long as the salt, as a whole, is pharmacologically acceptable.

Additional examples of pharmaceutically acceptable salts and their methods of preparation and use are presented in Handbook of Pharmaceutical Salts: Properties, Selection and Use (2002), which is incorporated herein by reference.

### II. Pharmaceutical Compositions

The present disclosure includes methods for treating metabolic dysfunction or conditions associated with metabolic dysfunction.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2-day to 12-week intervals, more usually from one to two week intervals.

In some embodiments, a pharmaceutical composition comprises a pharmaceutically acceptable carrier or excipient. The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

### III. Administration of Therapeutic Compositions

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, the composition is administered at a dose of between 1 mg/kg and 5000 mg/kg. In some embodiments, the composition is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

Certain aspects relate to the treatment and/or prevention of metabolic syndrome. In certain aspects, symptoms associated with metabolic syndrome, such as abdominal obesity, high blood pressure, high blood sugar, high serum triglycerides, and low serum high-density lipoprotein (HDL), are reduced or prevented. In certain aspects, administration of at least one composition disclosed herein to a patient results in at least one of: reduced waist circumference, reduced visceral fat, reduced insulin resistance, reduced blood pressure, reduced dyslipidemia, and/or reduced blood sugar levels, in the patient. In certain aspects, the administration of at least one composition disclosed herein to a patient results in the preservation of brown adipose tissue and/or the reduction of white adipose tissue.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Cannabidiol Preserves Brown Adipose Tissue During Progressive Metabolic Syndrome

To assess the effects of a CBD on BAT loss associated with progressive MetS, a cohort of 15-week-old Otsuka Long-Evans Tokushima Fatty (OLETF) rats were administered 200 mg CBD/kg by oral gavage for 5 weeks. Animals were fed ad libitum and monitored in metabolic cages alongside vehicle-treated OLETF and lean strain control Long-Evans Tokushima Otsuka (LETO) rats. Brown adipose tissue content was estimated from 2-dimensional images to quantify surface area. The Otsuka Long-Evans Tokushima Fatty (OLETF) rat is a monogenic model of diet-induced obesity accelerated by a mutation in the CCK receptor. These rats have a predictable, timed progression toward non-insulin dependent diabetes mellitus (T2DM) (>20 weeks), marked by a linear phase progression of hypertension (8-20 weeks), which closely resembles symptoms displayed by human T2DM symptoms including visceral adiposity, dyslipidemia and insulin resistance. At >40 weeks of age, OLETF rats suffer from severe metabolic dysfunction and therefore serve as a model of aged, severe MetS.

CBD preserved 55% (p<0.05) of BAT masses lost in OLETF control. Metabolomics revealed the most significant metabolite shift to be a 2.1-fold increase in ß-aminoisobutyric acid (BAIBA) (p<0.0001), which is associated with the browning of WAT.

The aspects herein show a chronic CBD regimen improves overall substrate metabolism via browning of WAT and/or preservation of BAT, which suggests a promising use for the prevention of impaired substrate metabolism progression after MetS onset.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Mascal M, Hafezi N, Wang D, Hu Y, Serra G, Dallas ML et al. Synthetic, non-intoxicating 8,9-dihydrocannabidiol for the mitigation of seizures. Sci Rep 2019; 9: 7778.
2. Assa-Glazer T, Gorelick J, Sela N, Nyska A, Bernstein N, Madar Z. Cannabis Extracts Affected Metabolic Syndrome Parameters in Mice Fed High-Fat/Cholesterol Diet. Cannabis Cannabinoid Res 2020; 5: 202-214.
3. Weiss L, Zeira M, Reich S, Har-Noy M, Mechoulam R, Slavin S et al. Cannabidiol lowers incidence of diabetes in non-obese diabetic mice. Autoimmunity 2006; 39: 143-151.
4. Ishida K, Mizuno A, Min Z, Sano T, Shima K. Which is the primary etiologic event in Otsuka Long-Evans Tokushima Fatty rats, a model of spontaneous non-insulin-dependent diabetes mellitus, insulin resistance, or impaired insulin secretion? Metabolism 1995; 44: 940-945.
5. Ewing LE, Skinner CM, Quick CM, Kennon-McGill S, McGill MR, Walker LA et al. Hepatotoxicity of a Cannabidiol-Rich Cannabis Extract in the Mouse Model. Molecules 2019; 24:1-17.
6. Remiszewski P, Jarocka-Karpowicz I, Biernacki M, Jastrza̧b A, Schlicker E, Toczek M, et al. Chronic cannabidiol administration fails to diminish blood pressure in rats with primary and secondary hypertension despite its effects on cardiac and plasma endocannabinoid system, oxidative stress and lipid metabolism. Int J Mol Sci 2020;21:1-24.
7. Kawano K, Hirashima T, Mori S, Natori T. OLETF (Otsuka Long-Evans Tokushima Fatty) rat: a new NIDDM rat strain. Diabetes Res Clin Pract 1994; 24 Suppl: S317-20.
8. Kawano K, Hirashima T, Mori S, Saitoh Y, Kurosumi M, Natori T. Spontaneous long-term hyperglycemic rat with diabetic complications: Otsuka Long-Evans Tokushima Fatty (OLETF) strain. Diabetes 1992;41:1422-8.
9. Rajesh M, Mukhopadhyay P, Btkai S, Patel V, Saito K, Matsumoto S, et al. Cannabidiol attenuates cardiac dysfunction, oxidative stress, fibrosis, and inflammatory and cell death signaling pathways in diabetic cardiomyopathy. J Am Coll Cardiol 2010;56:2115-25.
10. Ignatowska-Jankowska B, Jankowski MM, Swiergiel AH. Cannabidiol decreases body weight gain in rats: Involvement of CB2 receptors. Neurosci Lett 2011;490:82-4.
11. Huestis MA, Solimini R, Pichini S, Pacifici R, Carlier J, Busardò FP. Cannabidiol Adverse Effects and Toxicity. Curr Neuropharmacol 2019;17:974-89.
12. Thorwald MA, Godoy-Lugo JA, Rodriguez GJ, Rodriguez MA, Jamal M, Kinoshita H, et al. Nrf2-related gene expression is impaired during a glucose challenge in type II diabetic rat hearts. Free Radic Biol Med 2019;130:306-17.

What is claimed is:

1. A method of treating metabolic dysfunction or a condition associated with metabolic dysfunction comprising administering a therapeutically effective amount of a composition comprising CBD to an individual in need thereof.

2. The method of claim 1, wherein the condition associated with metabolic dysfunction is selected from obesity, insulin resistance, reduced glucose tolerance, and metabolic syndrome.

3. The method of claim 1 or 2, wherein the individual has been diagnosed with metabolic dysfunction or a condition associated with metabolic dysfunction.

4. The method of any of claims 1 to 3, wherein the method decreases body mass.

5. The method of claim 4, wherein the decrease in body mass comprises a reduction in abdominal fat.

6. The method of any of claims 1 to 5, wherein the method increases insulin receptor expression.

7. The method of any of claims 1 to 6, wherein the method reduces circulating adiponectin.

8. The method of any of claims 1 to 7, wherein the method increases circulating ghrelin.

9. The method of any of claims 1 to 8, wherein the method reduces plasma triglycerides.

10. The method of any of claims 1 to 9, wherein the method increases lipid metabolism.

11. The method of any of claims 1 to 10, wherein the CBD is a pharmaceutically acceptable salt, enantiomer, diastereomer, or prodrug thereof.

12. The method of any of claims 1 to 11, wherein the composition is administered orally, intraadiposally, intraarterially, intraarticularly, intracranially, intradermally, intralesionally, intramuscularly, intraperitoneally, intrapleurally, intranasally, intraocularally, intrapericardially, intraprostaticaly, intrarectally, intrathecally, intratumorally, intraumbilically, intravaginally, intravenously, intravesicularlly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

13. The method of any of claims 1 to 12, wherein the composition is administered to the subject at least two, three, four, five, six, seven, eight, nine or ten times.

14. The method of any of claims 1 to 13, wherein the CBD is administered at a dosage of at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, or 20 mg/kg.

15. A method of reducing body mass in an individual comprising administering to the individual a therapeutically effective amount of a composition comprising CBD.

16. The method of claim 15, wherein the reduced body mass comprises a reduction in abdominal fat.

17. The method of claim 15 or 16, wherein the method increases insulin receptor expression.

18. The method of any of claims 15 to 17, wherein the method reduces circulating adiponectin.

19. The method of any of claims 15 to 18, wherein the method increases circulating ghrelin.

20. The method of any of claims 15 to 19, wherein the method reduces plasma triglycerides.

21. The method of any of claims 15 to 20, wherein the method increases lipid metabolism.

22. The method of any of claims 15 to 21, wherein the CBD is a pharmaceutically acceptable salt, enantiomer, diastereomer, or prodrug thereof.

23. The method of any of claims 15 to 22, wherein the composition is administered orally, intraadiposally, intraarterially, intraarticularly, intracranially, intradermally, intralesionally, intramuscularly, intraperitoneally, intrapleurally, intranasally, intraocularally, intrapericardially, intraprostaticaly, intrarectally, intrathecally, intratumorally, intraumbilically, intravaginally, intravenously, intravesicularlly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

24. The method of any of claims 15 to 23, wherein the composition is administered to the subject at least two, three, four, five, six, seven, eight, nine or ten times.

25. The method of any of claims 15 to 24, wherein the CBD is administered at a dosage of at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, or 20 mg/kg.

26. A method of preserving brown adipose tissue in an individual comprising administering to the individual a therapeutically effective amount of a composition comprising CBD.

27. The method of claim 26, wherein the method increases ß-aminoisobutyric acid (BAIBA).

28. The method of claim 26 or 27, wherein the individual has, or is at risk of having, metabolic syndrome

29. The method of claim 26 or 28, wherein the CBD is a pharmaceutically acceptable salt, enantiomer, diastereomer, or prodrug thereof.

30. The method of any of claims 26 to 29, wherein the composition is administered orally, intraadiposally, intraarterially, intraarticularly, intracranially, intradermally, intralesionally, intramuscularly, intraperitoneally, intrapleurally, intranasally, intraocularally, intrapericardially, intraprostaticaly, intrarectally, intrathecally, intratumorally, intraumbilically, intravaginally, intravenously, intravesicularlly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

31. The method of any of claims 26 to 30, wherein the composition is administered to the subject at least two, three, four, five, six, seven, eight, nine or ten times.

32. The method of any of claims 26 to 31, wherein the CBD is administered at a dosage of at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, or 20 mg/kg.
